(19) United States
(12) Patent Application Publication
(10) Pub. No.: US 2007/0134184 A1
(43) Pub. Date: Jun. 14, 2007

(54) Organic therapeutic and cosmetic preparation

(76) Inventor: Stephen Pheiffer, Jim Thorpe, PA (US)

Correspondence Address: L. ZALE PATENT LAW OFFICE, 334 SOUTH FRANKLIN ST., WILKES-BARRE, PA, 18702, US

(21) Appl. No.: 11/638,122

(22) Filed: Dec. 13, 2006

Related U.S. Application Data
(60) Provisional application No. 60/597,624, filed on Dec. 13, 2005.

(51) Int. Cl. A61K 8/97

(52) U.S. Cl. 424/70.2; 424/74

(57) ABSTRACT

A mixture of an aqueous solution, essential oils and mechanically extracted kelp cytoplasm results in an emulsion with cosmetic and therapeutic properties. The preparation may be used on human and animal hair or skin. The moisturizing properties of the kelp cytoplasm provide the aqueous solution to the hair (keratin) which is absorbed, causing the hair to swell, and become heavier and more pliable. The alginate gels and oils in the emulsion coat the hair strands to reduce breakage and splitting and add weight to the strand. The preparation dries with gel capsules, which release moisture over time for a long-lasting effect. The preparation also provides nutrients, gel and moisture to the skin and fills micro cracks in the keratin layer of dry, chapped skin. The preparation seals off the cracks from the environment while trapping moisture pockets. This provides an environment conducive for healing.

### CROSS REFERENCES TO RELATED INVENTIONS

This application is a Continuation-In-Part of, and claims priority from U.S. Provisional Patent Application “Dermatological and Cosmetic Mist Spray-on Product Formulated from Freshly Harvested, Liquid Phaeophyte Kelp Cytoplasm” by Stephen Pheiffer, Ser. No. 60/597,624 filed Dec. 13, 2005, hereby incorporated by reference as if set forth in its entirety herein.

### BACKGROUND OF THE INVENTION

1. Field of the Invention

The present invention relates to a topical preparation which has both therapeutic and cosmetic properties.

2. Discussion of Related Art

Cosmetics are commonly used to add body, manageability and luster to a person's hair and/or keep it in place.

If the cosmetic is too light, i.e. with insufficient stiffness, it does not offer the appropriate hair holding properties.

If the cosmetic is too thick, i.e. with minimal flexibility, it would set the hair too firmly & stiff while leaving an unpleasant sticky or hard residue.

A growing number of consumers prefer that natural, organic materials be used in personal cosmetics since these cosmetics come in contact with the person or pet animal's skin and hair. Even though there are a number of synthetic materials which have been approved by the federal government, there is no guarantee that a consumer will have no adverse reaction to some of these synthetics after long-term use.

Some synthetic materials may be chemicals which have extreme pH levels or other unpleasant characteristics, which could negatively affect skin & hair protein.

Hair or animal's coat is primarily comprised of the protein keratin. If hair keratin becomes too dry, and has too little natural oils, the hair shaft becomes brittle and breaks or splits. The less oil, the less manageable and the less body it has. If hair or coat has too much natural oils, it obtains an undesirable greasy and matted appearance.

Aqueous solutions will keep the keratin pliable and increase the hair ‘body’. These, however, evaporate rapidly.

Many existing hair products incorporate at least some of the features listed above.

Human and animal skin is also covered with keratin. Therefore, many of the above mentioned properties also apply to skin of these entities.

Without the proper amount of oils the protective skin is subjected to drying and cracking. These micro cracks are openings in the protective skin, which allow microbes to enter & infect the cracks. This results in swelling, redness and pain. This is typically called chapped skin.

A person and animal would also be more exposed to harsh chemicals, irritants and allergies with such a cracked, permeable skin barrier.

Many existing skin and hand care products provide moisturizing and other agents which prevent chapping, and treat the cracking, irritations and infections. Many of these include synthetic ingredients.

In addition to drying, chapping, irritation and infection, there are skin disorders which cause symptoms similar to those described above. Eczema and Psoriasis are two examples of these. These conditions are fairly common yet have no effective, long term remedy. Not withstanding, there are medications which use synthetic ingredients which could have significant long-term unintentional side effects.

Currently, there is a need for a totally natural, 100% organic hair and skin preparation which offer effective, relief from dermatological irritations and, in addition, offers additional cosmetic benefits to the consumer.

### SUMMARY OF THE INVENTION

The present invention may be embodied as such a preparation for hair and skin treatment comprising:
a. 40 to 90% aqueous solution,
b. 0.05 to 2% oil,
c. 10 to 60% kelp cytoplasm containing a wide spectrum of nutrients and alginate gel and extracted by mechanical means to retain its natural properties, mixed with the aqueous mixture and the oil to provide an emulsion having cosmetic and therapeutic properties.

It may also be embodied as a method of creating a preparation for hair and skin treatment comprising the steps of:
a. filling a tank to a 40 to 90% volume with an aqueous solution,
b. stirring 0.05 to 2% oil into the aqueous solution,
c. stirring 10 to 60% kelp cytoplasm containing a wide spectrum of nutrients and alginate gel and extracted by mechanical means to retain its natural properties, mixed with the aqueous mixture and the oil to provide an emulsion having cosmetic and therapeutic properties.

It may also be embodied as a treatment for chronic skin-drying disorders comprising:
a. 40 to 90% aqueous solution,
b. 0.05 to 2% oil,
c. 10 to 60% kelp cytoplasm containing a wide spectrum of nutrients and alginate gel and extracted by mechanical means to retain its natural properties, mixed with the aqueous mixture and the oil to provide an emulsion having cosmetic and therapeutic properties.

### OBJECTS OF THE INVENTION

It is an object of the present invention to provide a cosmetic and therapeutic preparation for human and animal hair and skin.

It is an object of the present invention to provide a moisturizing preparation to reduce skin cracking and chapping.

It is an object of the present invention to provide a moisturizing preparation to reduce cracking and splitting of the hair shaft.

It is another object of the present invention to provide a preparation which is an anti-irritant.

It is another object of the present invention to provide a treatment for skin ailments such as eczema and psoriasis.

### BRIEF DESCRIPTION OF THE DRAWINGS

A complete understanding of the present invention may be obtained by reference to the accompanying drawings, when considered in conjunction with the subsequent detailed description, in which:

FIG. 1 is an illustration of a single strand of hair.

FIG. 2 is an illustration of hair treated with the preparation of the present invention.

FIG. 3 is an illustration of a healthy skin.

FIG. 4 shows dry, chapped skin.

FIG. 5 is diagram illustrating alginate gel contained in the kelp cytoplasm.

FIG. 6 shows the dry, chapped skin of FIG. 4 after the preparation of the present invention has been applied.

### DETAILED DESCRIPTION OF THE PRESENT INVENTION

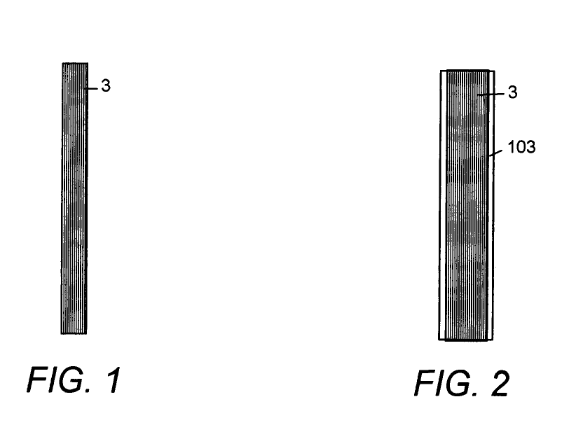

FIG. 1 is an illustration of a portion of a single strand of dry hair 3. It is very thin and lacks oils and moisture. A number of these strands will become very light, and hard to manage, typically referred to as “fly-away” hair.

FIG. 2 is an illustration of hair 3 treated with the preparation of the present invention. The keratin strand 3 absorbs the aqueous portion of the preparation (emulsion), causing it to expand in thickness. Strand 3 also becomes more pliable. In addition, the oils 103 and the alginate gel of the emulsion coat the outside of the hair strand 3, reducing the possibility that the strands 3 will crack or split. The oil 103 and alginate gel coating also adds shine and luster to the hair and coat.

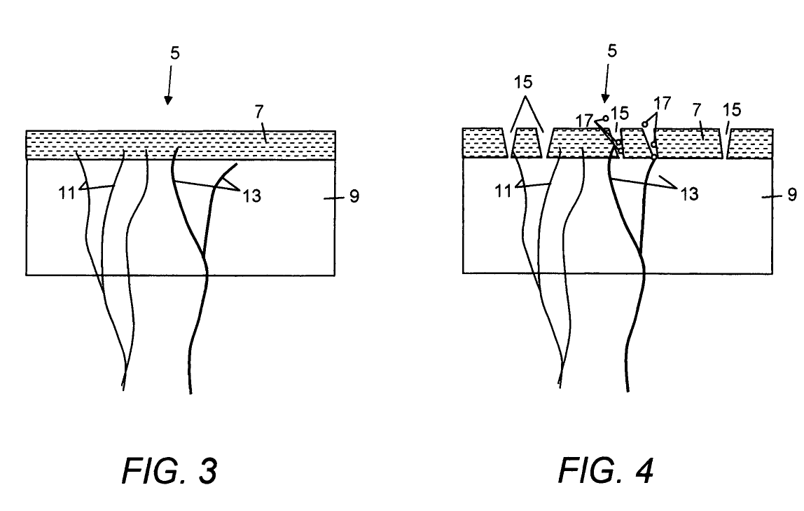

FIG. 3 is an illustration of a person's healthy skin 5. It shows a keratin layer 7, a living cellular layer 9 having nerve endings 11 and capillaries 13.

FIG. 4 shows dry, chapped skin 5. As moisture dries out of skin 5 and it loses its oils, skin 5 contracts and begins to form microcracks 15 through the keratin layer 7. Dry skin may result from the use of strong detergents which clean the oils off of a person's skin, allowing the skin to dry and chap.

The micro cracks 15 expose the cellular layer 9 and nearby nerve endings 11 and capillaries 13 to the outside world. Air-borne microbes 17 now are allowed to make their way into the capillaries 13.

Since the nerve endings 11 are also exposed, such skin areas would be more sensitive to any contact with caustic, acidic, or irritating environments. This will therefore increase the human and or animal discomfort level.

Kelp has certain known properties:

U.S. Pat. No. 7,025,966 Majmudar, “Compositions of Marine Botanicals to Provide Nutrition to Aging and Environmentally Damaged Skin”, Apr. 11, 2006 described kelp as a moisturizing agent.

In U.S. Pat. No. 6,974,799 Lintner, “Compositions Containing Mixtures of Tetrapeptides and Tripeptides”, Dec. 13, 2005, it stated that “Other thickening and gelling agents useful herein include . . . kelp”.

In U.S. Pat. No. 6,974,796 Girsh, “Therapeutic Compositions for Treatment of a Damaged Tissue”, it was mentioned that kelp is a good source of iodine useful in the rebuilding of tissue.

The preparation according to the present invention includes pure water which is absorbed by the keratin, essential oils, alginate gel and kelp cytoplasm which reduce cracking of the skin. The alginate gel also acts as an emulsifying agent holding the water and oils together in a stable emulsion.

In order for the kelp cytoplasm to perform these functions, it must retain its original natural properties. Therefore, the cytoplasm must be extracted from the kelp without chemically altering, denaturing or neutralizing the active organic molecules. The mechanical cell burst process applied to freshly harvested, crisp kelp yields filtered, non denatured liquid kelp cytoplasm which answers to these requirements.

Methods of Kelp Extraction and/or Preparation.

There are three common methods of cytoplasm extraction from kelp:

1. Hydrolyzing

This is a method utilizing an alkaline water medium to hydrolyze kelp seaweed with high temperatures into sludge form. After the kelp is heated & dehydrated into a dry ‘cake’, it is milled into a water-soluble powder. It is suggested that this processing technique is the most damaging to the natural organic components of fresh kelp cytoplasm.

2. Freezing

Fresh kelp is frozen and kept in a solid, frozen state prior to processing. The freezing technique expands water molecules within the cell membranes, rupturing the cell walls. This is then followed by high press rollers to separate the cytoplasm from the solid tissue. It is suggested that, as is the case with the above-mentioned method, the cytoplasm properties are negatively impacted at such extreme temperature levels.

The kelp cytoplasm used in the present invention employs the following method of extracting the cytoplasm from the kelp.

3. Mechanical Cell Bursting

The cells walls of crisp-fresh kelp are ruptured using mechanical impact and/or rapidly fluctuating mechanical pressures to release the cell's contents—the cytoplasm. This technology allows the kelp's organic cell contents to be released and, after filtration, be concentrated in the least damaging fashion, resulting in liquid kelp cytoplasm which contains all the kelp's original properties.

The kelp cytoplasm used in the present invention is extracted using mechanical cell bursting technology of Phaeophyte kelp, Ecklonia Maxima. The kelp cytoplasm contains alginate gel which traps aqueous solutions in a plurality of capsules. Moisture evaporation rate is reduced by presence of the alginate gel.

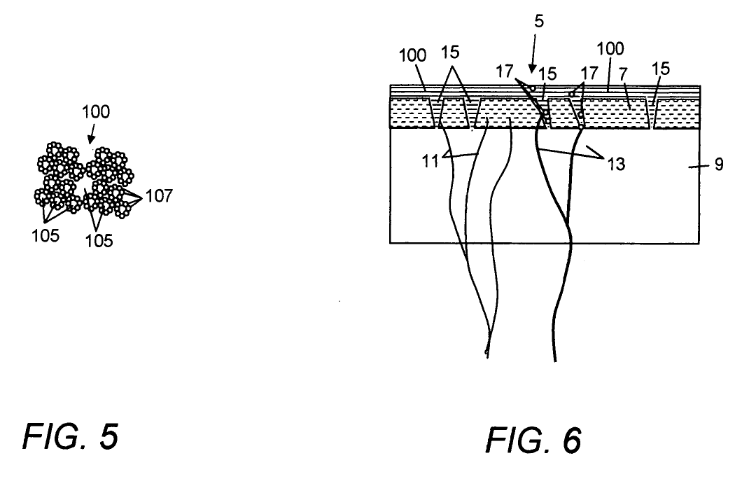

FIG. 5 is a diagram illustrating alginate gel in the kelp cytoplasm. It is believed that moisture is trapped in pockets 105 when molecules 107 (shown as circles here) of the treatment 100 encircle water or an aqueous solution. The result is a gel-like state which holds water so that it does not readily evaporate, but is able to slow-release its water content over an extended time period.

The kelp cytoplasm carries alginate gel which encapsulates moisture and supplies it to the hair shaft resulting in absorption causing the hair shaft expansion. The oils and the alginate gels in the emulsion act to treat the strand to reduce breakage and splitting.

FIG. 6 shows the dry, chapped skin 5 of FIG. 4 after the preparation 100 of the present invention has been applied. Preparation 100 permeates and fills the microcracks 15.

Since the preparation 100 fills the cracks, it seals off from the microbes 17.

The preparation 100 of the present invention is made from kelp cytoplasm; it has a similar tonicity to that of the cytoplasm of our cells.

A high pH (alkaline) dissolves the keratin layer. A low pH denatures the keratin protein. Neither of these environments are conducive to healing.

The preparation 100 of the present invention has a pH similar to that of our natural internal body fluids.

The exposed portion of the preparation 100 includes alginate gel which, after the mist-on application, offers a protective gel membrane covering the skin or hair, sealing in the moisture and promoting trans-dermal absorption of the natural nutrients contained in the kelp cytoplasm. For these and other reasons, the preparation 100 of the present invention provides an environment conducive for healing.

Process for Manufacturing
A tank is filled to approximately 70% of its capacity with water.
Approximately 0.1% to 1% of the tank volume of essential oils are added to the water. For example, these oils may be Rosemary oil, Lavender oil and/or Tee Tree oil.
The water and essential oils are stirred with gentle agitation, to maintain homogeneous oil dispersion in the tank.
A 30% volume of mechanically extracted, fresh kelp cytoplasm is then added to the tank. (Preferably Ecklonia maxima sea kelp.)
The contents of the tank are gently and frequently stirred to create an emulsion. The emulsion is ready to be filled into bottles while maintaining the stirring action in the tank.

This results in a natural preparation which is beneficial to human and animal hair and skin.

In an alternative embodiment, an oxygenating agent may be added to the preparation. The oxygenating agent offers shelf-life stability to the natural preparation and provides free oxygen which is dissolved into the solution. Preferably this oxygenating agent is made from natural ingredients. One such oxygenating agent is a mixture of acetic acid (fermented sourced), hydrogen peroxide (50% concentration) and water in a 1:1:2 ratio. This provides free oxygen which is dissolved in the aqueous portion of the preparation.

Upon spraying the preparation onto the person or animal, it releases the dissolved oxygen. Therefore, spraying the preparation on the face of the user provides a pleasant mixture of the preparation and released oxygen. This provides certain desirable and aesthetic qualities of the product.

It is the viability of these unaltered kelp cytoplasm properties in this preparation that offer the following observed dermatological and cosmetic benefits:
(1) Cosmetics—hair & skin care:
   Improves hair density, body and shine
   Improves the condition of damaged hair
   Moisturizes dry skin
   Rejuvenates, tightens and improves skin texture
(2) Dermatological:
   Alleviates certain dermatological irritations and disorders such as eczema, psoriasis, dandruff in humans and animals
   Rapidly alleviates and soothes sunburn
   Rapidly alleviates various skin itch conditions
   Facilitates healing

While several presently preferred embodiments of the novel invention have been described in detail herein, many modifications and variations will now become apparent to those skilled in the art.

What is claimed is:

1. A preparation for hair and skin treatment comprising:
a. 40 to 90% aqueous solution,
b. 0.05 to 2% oil,
c. 10 to 60% kelp cytoplasm containing a wide spectrum of nutrients and alginate gel and extracted by mechanical means to retain its natural properties, mixed with the aqueous mixture and the oil to provide an emulsion having cosmetic and therapeutic properties.

2. The preparation for hair and skin treatment of claim 1 further comprising: an oxygenating agent.

3. The preparation for hair and skin treatment of claim 1 wherein:
the oxygenating agent includes acetic acid and hydrogen peroxide.

4. The preparation for hair and skin treatment of claim 1 wherein the kelp cytoplasm is mechanically extracted without the application of any heat, freezing, dehydration and/or significant changes to its pH.

5. A method of creating a preparation for hair and skin treatment comprising the steps of:
a. filling a tank to a 50 to 80% volume with an aqueous solution,
b. stirring 0.05 to 2% oil into the aqueous solution,
c. stirring 20 to 40% kelp cytoplasm extracted by mechanical means to retain its natural properties into the aqueous solution and the oil to provide an emulsion having cosmetic and therapeutic properties.

6. The method of creating a preparation for hair and skin treatment of claim 5 further comprising the step of:
adding an oxygenating agent.

7. The method of creating a preparation for hair and skin treatment of claim 5 wherein the step of adding an oxygenating agent comprises the steps of:
adding acetic acid to the mixture, and
adding hydrogen peroxide.

8. The preparation for hair and skin treatment of claim 1 wherein the kelp cytoplasm is mechanically extracted without the application of any heat, freezing, dehydration and/or significant changes to its pH.

9. A treatment for chronic skin-drying disorders comprising:
a. 40 to 90% aqueous solution,
b. 0.05 to 2% oil,
c. 10 to 60% kelp cytoplasm containing a wide spectrum of nutrients and alginate gel and extracted by mechanical means to retain its natural properties, mixed with the aqueous mixture and the oil to provide an emulsion having cosmetic and therapeutic properties.

10. The treatment for chronic skin-drying disorders of claim 9 further comprising:
an oxygenating agent.

11. The treatment for chronic skin-drying disorders of claim 9 wherein:
the oxygenating agent includes acetic acid and hydrogen peroxide.

12. The treatment for chronic skin-drying disorders of claim 9 wherein:
the kelp cytoplasm is mechanically extracted without the application of any heat, freezing, dehydration and/or significant changes to its pH.

13. The treatment for chronic skin-drying disorders of claim 9 wherein:
the skin-drying disorder is eczema.

14. The treatment for chronic skin-drying disorders of claim 9 wherein:
the skin-drying disorder is psoriasis.

15. The treatment for chronic skin-drying disorders of claim 9 wherein:
the skin-drying disorder is dandruff.
